(19) United States
(12) Patent Application Publication
(10) Pub. No.: US 2017/0251968 A1
(43) Pub. Date: Sep. 7, 2017

(54) A method and system for detecting visual attention

(71) Applicant: Beijing University of Technology, Beijing (CN)

(72) Inventors: Mi Li, Beijing (CN); Shengfu Lu, Beijing (CN); Jing Wang, Beijing (CN); Ning Zhong, Beijing (CN)

(21) Appl. No.: 15/324,677

(22) PCT Filed: Mar. 5, 2015
(86) PCT No.: PCT/CN2015/073706

§ 371 Date: Jan. 6, 2017

(30) Foreign Application Priority Data
Sep. 1, 2014 (CN) 201410441421.8

(51) Int. Cl. A61B 5/16; A61B 3/00; A61B 3/113; A61B 3/11; A61B 5/00

(52) U.S. Cl. CPC A61B 5/168; A61B 3/112; A61B 5/7225; A61B 3/113; A61B 3/0025

(57) ABSTRACT

The present disclosure discloses a method and system for detecting visual attention. Embodiments of the present disclosure collect time sequences and corresponding pupil diameter sequences of each point of regard during visual attention process. Attention change curve is plotted according to the corresponding relationship between time sequences and pupil diameter sequences. The attention change curve is divided into four stages based on pre-setting time parameters and pupil diameter parameters. Numerical analysis of each stage is finished based on duration and rate of pupil change. Quantitative calculation of visual attention of each stage is realized, and the changing procedure of visual attention can be portrayed entirely, systematically and quantitatively.

### CROSS REFERENCE TO RELATED APPLICATION

This application is a national stage application of International application number PCT/CN2015/073706 filed Mar. 5, 2015, titled “A method and system for detecting visual attention,” which claims the priority benefit of Chinese Patent Application No. 201410441421.8, filed on Sep. 1, 2014, which is hereby incorporated by reference in its entirety.

### TECHNICAL FIELD

The present disclosure involves cognitive science, cognitive psychology, and quantitative analysis areas, and particularly to a method and system for detecting visual attention.

### BACKGROUND

As everyone knows, eyes are windows to the soul. It is an important way to obtain outside information. The human visual process depends on visual channel largely. Some research shows that human obtains 80%-90% outside information from eyes. Therefore, it is considered that research on eyes-movement is the most effective ways to study human cognitive such as attention, memory, inference and reading. Research of human cognitive such as attention, memory, inference and reading through eye-movement was studied in the 19^{th} century. In recent years, some precision eye-movement instruments provide an effective instrument for studying the relationship between eye-movement and human cognitive (attention, memory, inference and reading) through analyzing recorded eye-movement data.

However, current research on human cognitive usually uses qualitative description, which lacks quota portray, integrity, dynamics and quantitative characterization.

### SUMMARY

The present disclosure discloses a method and system for detecting visual attention. It realizes quantitative calculation of each stage of visual attention, which portrays the attention change process completely, systematically and quantificationally.

The present disclosure discloses a method and system for detecting visual attention. Some implementations herein are provided as follow.

Time sequences and corresponding pupil diameter sequences of each regarding point during visual attention process are collected. Attention change curve is plotted according to the corresponding relationship between time sequences and pupil diameter sequences. The attention change curve is divided into four stages based on pre-setting time parameters and pupil diameter parameters. Numerical analysis of each stage is finished based on duration and rate of pupil change.

The process of dividing the change curve into four attention stages may include the following steps. Five attention points of the change curve are selected based on pre-setting time parameters and pupil diameter parameters. The attention change curve is divided into four attention stages based on five attention points. These five attention points are: an attention starting point, a minimum point of pupil diameter during the attention process, a maximum point of pupil diameter during attention process, an attention ending point and a stationary point of pupil diameter after attention process; the four attention stages are: an attention preparation stage, an attention processing stage, an attention maintaining stage and a dissolution stage.

Numerical analysis of attention preparation stage including the following steps:

Attention preparation time and attention preparation rate is calculated,

t_{AB}=t_{B}−t_{A},

R_{AB}=(P_{B}−P_{A})/(t_{B}−t_{A}),

t_{A} represents the moment of attention starting point; P_{A} is the value of the pupil diameter at t_{A}, t_{B} represents the moment of the attention preparation ending point, P_{B} is the value of the pupil diameter at t_{B}, t_{AB} represents attention preparation time, and R_{AB} represents the attention preparation rate.

Numerical analysis of attention processing stage including the following steps:

Attention processing time and attention processing rate is calculated,

t_{BC}=t_{C}−t_{B},

R_{BC}=(P_{C}−P_{B})/(t_{C}−t_{B}),

t_{B} represents the moment of attention preparation ending point which is the moment of the attention processing starting point, P_{B} is the value of the pupil diameter at t_{B}, t_{C} represents the moment of attention processing ending point, P_{C} is the value of the pupil diameter at t_{C}, t_{BC} represents the attention processing time, R_{BC} represents the attention processing rate.

Numerical analysis of attention maintaining stage including the following steps:

Attention maintaining time and attention change rate is calculated,

t_{CD}=t_{C}−t_{C},

R_{CD}=P_{CD}/(t_{D}−t_{C}),

t_{C} represents the moment of attention processing ending point which is the moment of attention maintaining starting point; t_{D} represents the moment of attention maintaining end point; t_{CD} represents attention maintaining time,

P_{CD}=Σᵢ₌₁ⁿPᵢ/n, i=1, 2, 3, . . . , n, Pᵢ represents pupil diameter of No. i attention point of the attention maintaining stage; R_{CD} represents the attention change rate.

Numerical analysis of attention dissolution stage including the following steps:

Attention recovery time and attention recovery rate are calculated,

t_{DE}=t_{E}−t_{D},

R_{DE}=(P_{E}−P_{D})/(t_{E}−t_{D}),

t_{D} represents the moment of the attention maintaining ending point, t_{E} represents the moment of dissolution recovery finishing point; t_{DE} represents the attention recovery time; P_{D} is the value of the pupil diameter at t_{D}, P_{E} is the value of the pupil diameter at t_{E}, R_{DE} represents the attention recovery rate.

As mentioned above, according to the visual attention detection method provide by the present disclosure, attention process can be expressed by attention change curve dynamically and systematically. The attention dynamic change and the attention change curve are compared to study attention process in detail; attention regularity is portrayed more entirely and systematically. While at the same time, the quantitative calculation for each stage of attention in the present disclosure portray the attention change process completely, systematically and quantificationally which provides a new viewpoint and method. Quota portrays, integrality and dynamic way are used to provide a new method for cognitive science and cognitive psychology research.

The present disclosure also provides a detection system for the visual attention, which can proceed quantitative calculation for each stage of attention. The change process is portrayed entirely, systematically and quantificationally.

A system for detecting visual attention may include the following components:

Stimulation providing device 1 is used for setting visual stimulation task and providing to the users. Eye-movement collecting device 2 is used for time sequences and corresponding pupil diameter sequences of visual attention. Attention change curve generation device 3 connects with eye-movement collecting device 2, which is used for generating attention change curve based on the corresponding relationship between time sequences and pupil diameter sequences. Numerical analysis device 4 connects with attention change curve generation device 3, which is used for numerical analysis of change curve based on a time duration and a pupil diameter change rate.

The attention change curve generation device 3 takes time sequences as the horizontal axis and pupil diameter sequence as a vertical axis to generate attention change curve.

A system for detecting visual attention may include: an attention change curve is processed by the numerical analysis device (4) with the following steps: five attention points are selected according to pre-setting time parameters and pupil diameter parameters. Attention change curve is divided into four stages based on the five attention points. These five attention points are: an attention starting point, a minimum point of pupil diameter during the attention process, a maximum point of pupil diameter during attention process, an attention ending point and stationary point of pupil diameter after attention process; the four attention stages are: an attention preparation stage, an attention processing stage, an attention maintaining stage, and a dissolution stage.

A system for detecting visual attention may include: numerical analysis is processed of by numerical analysis device (4) with the following steps: an attention preparation time and an attention preparation rate of the attention preparation stage are calculated. The attention processing time and the attention processing rate of the attention processing stage are calculated. The attention maintaining time and attention change rate of the attention maintaining stage are calculated. The attention recovery time and attention recovery rate of the attention dissolution stage are calculated.

The attention detection system provided by the present disclosure expresses attention process dynamically and systematically by the attention change curve. The attention dynamic change and attention change curve are compared to study attention process in detail; attention regularity is portrayed more completely and systematically. While at the same time, the quantitative calculation for each stage of attention in the present disclosure portray the attention change process entirely, systematically and quantificationally which provides a new viewpoint and method. Quota portrays, integrality and dynamic way are used to provide a new method for cognitive science and cognitive psychology research.

### BRIEF DESCRIPTION OF THE DRAWINGS

FIG. 1 is a flowchart illustrating a process of detecting visual attention based on preferred embodiments of the disclosure.

FIG. 2 is a flowchart illustrating a process of determining an attention change curve based on preferred embodiments of the disclosure.

FIG. 3 illustrates a user attention change curve based on preferred embodiments of the disclosure.

FIG. 4 is a structure diagram illustrating a system for detecting visual attention based on preferred embodiments of the disclosure.

### DETAILED DESCRIPTION OF THE PREFERRED EMBODIMENTS

The present disclosure will be described in more detail accompanied with the preferred embodiments. These embodiments are just examples so that the protective scope is not limited by it. Description of general structure and technology is omitted to avoid confusing principles of the present disclosure.

When analyzing human attention process, it is widely believed that attention includes four subcomponents: attentional orienting, selective attention, divided attention and sustained attention. Attention network theory is disclosed based on quantities cognitive psychology and biological research. It is considered, at cognitive, neural network and neurotransmitter level, that attention is divided into three attention networks: vigilance of executing attention, orientation of executing attention and execution controlling function. Vigilance means realizing and sustaining a vigilance state; orientation means selecting information from the cognitive input; execution controlling means solving the conflict between responses. Accordingly, attention is not integral and can be refined.

As for the situation above, this present disclosure refines attention and generate an attention change curve through eye-movement data recorded by the eye-movement collecting device.

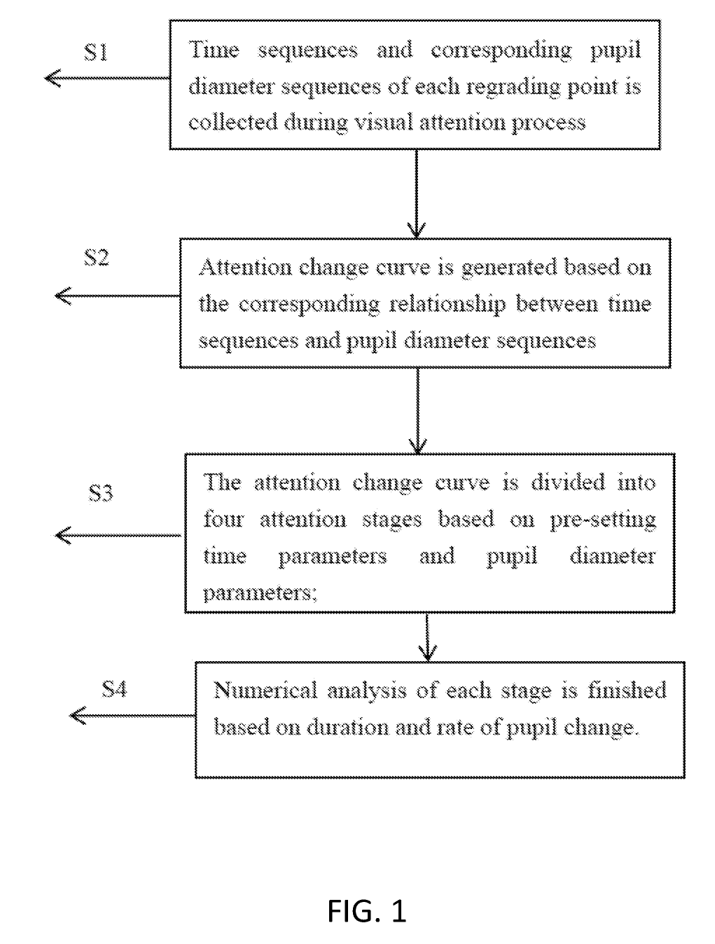

FIG. 1 is a flowchart illustrating a process of detecting visual attention based on preferred embodiments of the disclosure.

Step S1, time sequences and corresponding pupil diameter sequences of each regarding point during visual attention process are collected.

It's important to note that change of attention leads to the change of pupil diameter during attention process. Attention change curve is quantitatively expressed by using a pupil diameter curve in the present disclosure, dynamic change features of attention can be expressed by dynamic change features of the pupil diameter.

Based on the theory above, the visual stimulation task that leads to the pupil diameter change is set at the beginning of S1, including the following steps:

At first, 4 pictures are randomly selected from picture-base. Since the size, brightness and gray level of these four pictures are different, these pictures should be normalized. In the present disclosure, each picture is normalized with the same size, brightness and gray level by image processing software such as Photoshop®. The size of the normalized picture can be 200*200 pixel.

Then, 4 normalized pictures are combined tightly according to 4 quadrants without overlap to form a visual stimulation task.

Next, this visual stimulation task is showed to the user. In some embodiments, first, 5 seconds blank screen is showed to the user (“+” on the century of blank screen); then visual stimulation task appears in the century of blank screen (horizontal view and vertical view are all 12°), let the user watch 5 seconds; at last, 5 seconds blank screen is showed to the user (“+” on the century of blank screen). During this process, time sequence t₁, t₂ . . . tₙ and corresponding pupil diameter sequence P₁, P₂ . . . Pₙ of each regarding point is synchronously collected by the eye-movement collecting device. “n” represents the number of regarding points, P₁ is the value of the pupil diameter at t₁, P₂ is the value of the pupil diameter at t₂, the rest can be done in the same manner.

Tobii T120 non-invasive eye tracker is used in preferred embodiments of the present disclosure, and the attention change curve when users watch pictures is recorded with 120 Hz sampling frequency.

Step S2, the attention change curve is plotted according to the corresponding relationship between time sequences and pupil diameter sequences.

In some embodiments, pupil diameter vs. time curve is plotted to take time sequence as the horizontal axis and pupil diameter sequence as the vertical axis. It is the attention change curve of the user.

Step S3, the attention change curve is divided into four stages based on pre-setting time parameters and pupil diameter parameters.

In some embodiments, five attention points of the change curve are selected based on pre-setting time parameters and pupil diameter parameters. Pre-setting time parameters includes attention starting point and attention ending point. Pre-setting pupil diameter parameter includes minimum point of pupil diameter during attention process, the maximum point of pupil diameter during attention process and stationary point of pupil diameter after attention process.

In the preferred embodiments of the present disclosure, these five attention points are attention starting point, the minimum point of pupil diameter during attention process, the maximum point of pupil diameter during attention process, attention ending point and stationary point of the pupil diameter after the attention process.

Then, the process between any two adjacent attention points is taken as one attention stage to divide the attention curve into four attention stages. These four attention stages include an attention preparation stage, an attention processing stage, an attention maintaining stage, and an attention dissolution stage.

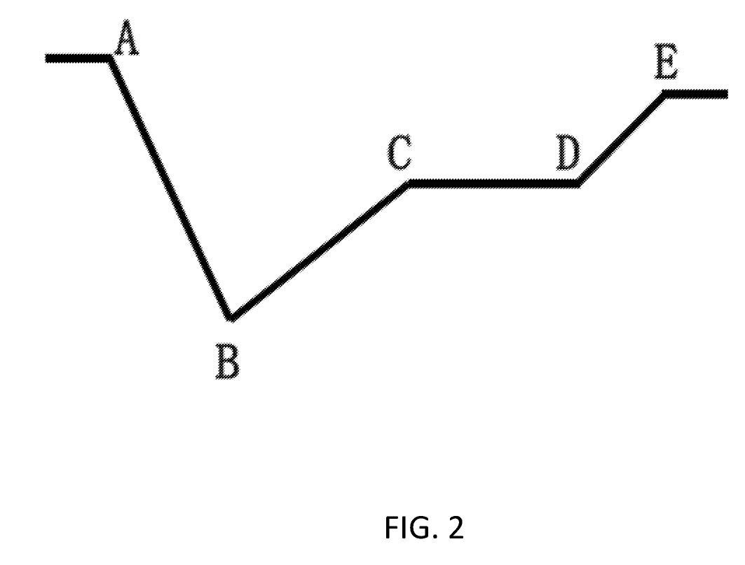

FIG. 2 is a flowchart illustrating a process of determining an attention change curve based on preferred embodiments of the disclosure.

Next, the description of selecting four attention stages from 5 change curve points is given which is shown in FIG. 2 and S2.

From FIG. 2, the change curve may include five attention points (A, B, C, D, E) and 4 attention stages (AB, BC, CD, DE).

A: attention starting point

B: minimum point of pupil diameter during attention process

C: maximum point of pupil diameter during attention process

D: attention ending point

E: stationary point of pupil diameter after attention process

These five attention points (A, B, C, D, E) divide change curve into four attention stages.

AB stage: attention preparation stage that includes attention vigilance and attention orientation.

BC stage: attention processing stage that includes selection attention, processing attention, and transformation attention. Attention processing process is to select an attention at first. Then this selected attention is processed, and then attention is transformed to the next selected attention. These three attention components are circulated like this until all the attention is processed of.

CD stage: attention maintaining stage. After finishing of attention processing stage, the attention is maintained until attention end.

DE stage: attention dissolution stage that is attention recovery stage. It is the tranquillization stage from maintaining recovery to no attention.

Step S4, numerical analysis of each stage is finished based on duration and rate of pupil change. What the details of the numerical analysis of four stages mention in step S3 are described in below respectively.

This description will be given based on the attention change curve which may include five attention points (A, B, C, D, E) and 4 attention stages (AB, BC, CD, DE) which are shown in FIG. 2.

Attention preparation stage: attention preparation is the first stage of attention corresponding to line AB where pupil diameter is changing; A is attention starting point, and also the starting point of attention preparation, and B is the ending point of attention preparation and also the starting point of attention processing stage. In the present disclosure, attention preparation time t_{AB} and attention preparation rate R_{AB} are established for describing attention preparation stage, which is shown in formula (1).

Numerical analysis of attention preparation stage including the following steps:

Attention preparation time and attention preparation rate is calculated,

t_{AB}=t_{B}−t_{A},

R_{AB}=(P_{B}−P_{A})/(t_{B}−t_{A}), (1)

t_{A} represents the moment of attention starting point A, P_{A} is the value of the pupil diameter at t_{A}, t_{B} represents the moment of the attention preparation ending point B, P_{B} is the value of the pupil diameter at t_{B}, t_{AB} represents the attention preparation time, R_{AB} represents the attention preparation rate, which is the slope of line AB, R_{AB}<0.

Attention preparation stage is a vigilance process of attention with attention orientation. Attention vigilance is the process of no attention state transforming into attention state; attention orientation is the continuous ensure state of attention state. Pupil diameter is large under a no attention state and is small under an attention state. Thus, the pupil diameter is adjusted from large to small little by little during an attention preparation stage to prepare for attention processing. As for an attention preparation rate R_{AB}<0, the absolute value of R_{AB} represents the speed of attention preparation. When R_{AB}<0, it means pupil diameter is shrinking during attention preparation.

Attention processing stage: attention processing is the second stage of attention corresponding to line BC where pupil diameter is changing. B is the ending point of attention preparation and also the starting point of attention processing stage, C is the finishing point of attention processing and also the starting point of attention maintaining stage. In the present disclosure, attention processing time t_{BC} and attention processing rate R_{BC} are established for describing attention processing stage which is shown in formula (2).

Numerical analysis of attention processing stage including the following steps:

Attention processing time and attention processing rate is calculated,

t_{BC}=t_{C}−t_{B},

R_{BC}=(P_{C}−P_{B})/(t_{C}−t_{B}), (2)

t_{B} represents the moment of attention processing starting point B, P_{B} is the value of the pupil diameter at t_{B}, t_{C} represents the moment of attention processing ending point, P_{C} is the value of the pupil diameter at t_{C}, t_{BC} represents attention processing time, R_{BC} represents attention processing rate which is the slope of line BC, R_{BC}>0.

The attention processing stage includes three attention components, which are: selection attention, processing attention, and transformation attention. Mental load becomes larger with the increasing of attention processing, and pupil diameter also increases from the beginning of attention processing (P_{B}) to the end of attention processing (P_{C}). Thus, the attention processing rate R_{BC} is greater than zero.

Attention maintaining stage: attention maintaining is the third stage of attention corresponding to line CD where pupil diameter is changing. C is the starting point of attention maintaining stage and also the finishing point of attention processing, and D is the ending point of attention maintaining and also the starting point of attention dissolution stage. In the present disclosure, attention maintaining time t_{CD} and attention changing rate R_{CD} are established for describing attention maintaining stage which is shown in formula (3).

Numerical analysis of attention maintaining stage including the following steps:

Attention maintaining time and attention changing rate is calculated,

t_{CD}=t_{D}−t_{C},

R_{CD}=P_{CD}/(t_{D}−t_{C}), (3)

t_{C} represents the moment of attention maintaining starting point C, t_{D} represents the moment of attention maintaining ending point, t_{CD} represents the attention maintaining time, P_{CD}=Σᵢ₌₁ⁿPᵢ/n i=1, 2, 3, . . . , n; Pᵢ represents pupil diameter of No. i attention point of attention maintaining stage; R_{CD} represents attention changing rate.

Attention maintaining stage reflects attention maintaining ability, which means attention. Attention changing rate R_{CD} means the ratio between average pupil diameter value during the attention maintaining and the attention maintaining time. The lower the R_{CD} is, the higher the attention is.

Attention dissolution stage: attention dissolution is the fourth stage of attention which means attention recovery process, attention from attention maintaining recovery to no attention state. It corresponds to line DE where pupil diameter is changing. D is the ending point of attention maintaining and also the starting point of attention dissolution stage, E represents the attention dissolution recovery finishing point. In the present disclosure, attention recovery time t_{DE} and attention recovery rate R_{DE} are established for describing attention dissolution stage which is shown in formula (4).

Numerical analysis of attention dissolution stage including the following steps:

Attention recovery time and attention recovery rate is calculated,

t_{DE}=t_{E}−t_{D},

R_{DE}=(P_{E}−P_{D})/(t_{E}−t_{D}), (4)

t_{D} represents the moment of attention dissolution starting point D, t_{E} represents the moment of dissolution recovery finishing point E, t_{DE} represents attention recovery time, P_{D} is the value of the pupil diameter at to, P_{E} is the value of the pupil diameter at t_{E}, R_{DE} represents the attention recovery rate, which is the slope of line DE, R_{DE}>0.

Attention dissolution stage is an attention recovery process, and attention recovers from attention maintaining recovery to no attention state. Pupil diameter also increases from the attention maintaining (P_{D}) to no attention state (P_{E}). Thus, the attention pupil diameter changing rate R_{DE} is greater than zero.

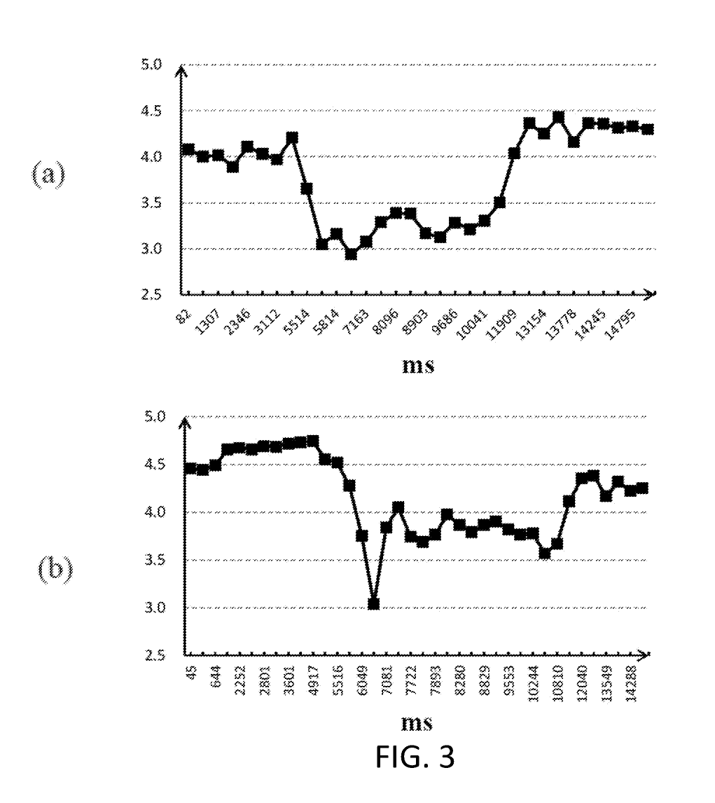

FIG. 3 illustrates a user attention change curve based on preferred embodiments of the disclosure.

(a) In FIG. 3 is attention change curve of user 1, (b) in FIG. 3 is attention change curve of user 2 which are shown in FIGS. 3(a) and (b). The method of detecting visual attention of the present disclosure is described based on the attention change curve of user 1 and user 2. Division mode (the change curve is divided into five attention points A, B, C, D, E; 4 attention stages) in step S3 and FIG. 2 is used to label attention change curve of user 1 and user 2. According to the time and pupil diameter parameters corresponding to five attention points and four attention stages in FIGS. 3(a) and (b), numerical analysis method in step S4 is used for each attention stage; numerical analysis results are obtained. Table 1 shows the required time and attention feature in each attention stage of user 1 and user 2.

TABLE 1

| | t_{AB} | | t_{BC} | | t_{CD} | | t_{DE} | |
|---|---|---|---|---|---|---|---|---|
| | (ms) | R_{AB} | (ms) | R_{BC} | (ms) | R_{CD} | (ms) | R_{DE} |
| User 1 | 1982 | −0.06 | 1515 | 0.03 | 1411 | 0.23 | 3113 | 0.03 |
| User 2 | 766 | −0.20 | 1215 | 0.08 | 3171 | 0.12 | 2830 | 0.03 |

In table 1, from attention preparation stage, attention preparation time of user 1 (t_{AB}=1982 ms) is higher than user 2 (t_{AB}=766 ms), the absolute value of attention preparation rate of user 1 (|RAB|=0.06) is lower than user 2 (|RAB|=0.20) which means the attention preparation efficiency of user 1 is lower than user 2.

From attention processing stage, attention processing time of user 1 (t_{BC}=1515 ms) is higher than user 2 (t_{BC}=1215 ms), the value of attention processing rate of user 1 (R_{BC}=0.03) is lower than user 2 (R_{BC}=0.08), which means the attention processing efficiency of user 1 is lower than user 2.

From attention maintaining stage, the attention maintaining time of user 1 (t_{CD}=1411 ms) is lower than user 2 (t_{CD}=3171 ms), the value of attention changing rate of user 1 (R_{CD}=0.23) is higher than user 2 (R_{CD}=0.12); the more the attention changes, the greater effort for maintaining attention will be done. It means attention of user 1 is lower than user 2.

From attention processing stage, attention processing time of user 1 (t_{DE}=3113 ms) is higher than user 2 (t_{DE}=2830 ms), and the value of attention recovery rate of user 1 (R_{DE}=0.03) is same with user 2 (R_{BC}=0.03) which means the attention dissolution efficiency of user 1 is lower than user 2.

In general, based on the comparison of each stage, the attention ability of user 1 is lower than user 2.

Based on the method of detecting visual attention in preferred embodiments of the present disclosure, attention process is expressed by attention change curve dynamically and systematically. It compares attention dynamic change and attention change curve to study attention process in detail; attention regularity is portrayed more completely and systematically. While at the same time, the quantitative calculation for each stage of attention in the present disclosure portray the attention change process entirely, systematically and quantificationally which provides a new viewpoint and method. It uses quota portray, integrality and dynamic way to provide a new method for cognitive science and cognitive psychology research.

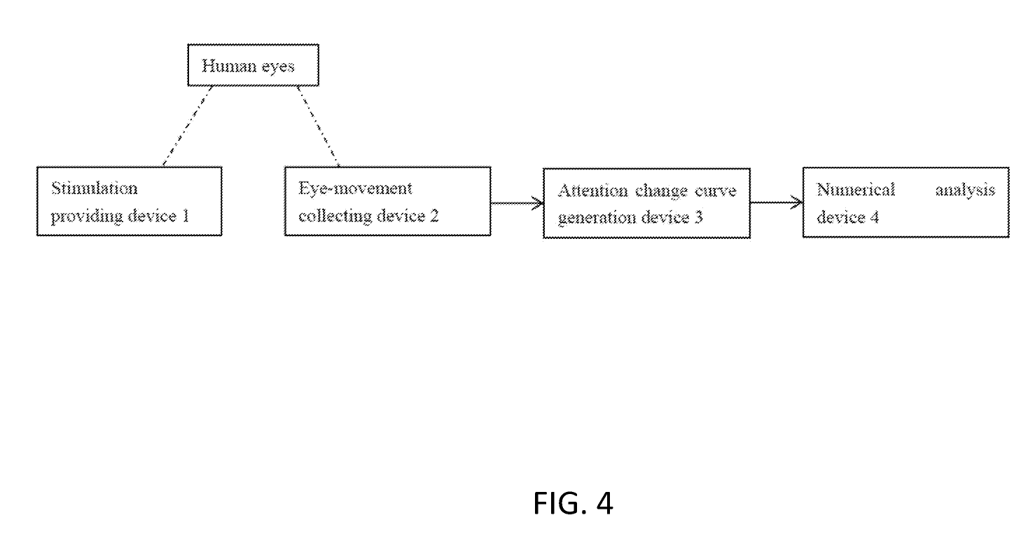

FIG. 4 is a structure diagram illustrating a system for detecting visual attention based on preferred embodiments of the disclosure.

The system for detecting visual attention in preferred embodiments of the present disclosure comprising: stimulation providing device 1, eye-movement collecting device 2, attention change curve generation device 3, numerical analysis device 4.

It is important to note that attention change leads to the change of pupil diameter.

Attention change curve can be quantitatively expressed by pupil diameter curve, and dynamic change features of attention change curve can be expressed by dynamic change features of pupil diameter curve.

In some embodiments, visual stimulation task which leads to pupil diameter change is set by stimulation providing device 1, and it is provided to the user. Stimulation providing device 1 select 4 pictures from picture-base randomly. Since the size, brightness and gray level of these four pictures are different, these pictures should be normalized. In the present disclosure, each picture is normalized with same size, brightness and gray level by image processing software such as Photoshop. The size of the normalized picture can be 200*200 pixel.

Then, 4 normalized pictures are combined tightly according to 4 quadrants without overlap to form a visual stimulation task.

Next, this visual stimulation task is shown to the user by stimulation providing device 1. In some embodiments, first, 5 seconds blank screen is shown to the user (“+” on the century of blank screen); then visual stimulation task appears in the century of blank screen (horizontal view and vertical view are all 12°), let the user watch 5 seconds; at last, 5 seconds blank screen is showed to the user (“+” on the century of blank screen).

It can be seen that device with picture selection, processing and display functions can be used as stimulation providing device 1.

Eye-movement collecting device 2 is used for collecting time sequences and corresponding pupil diameter sequences of visual attention.

Time sequence t₁, t₂ . . . tₙ and corresponding pupil diameter sequence P₁, P₂ . . . Pₙ of each regarding point is synchronously collected by the eye-movement collecting device.

“n” represents the number of regarding points, P₁ is the value of the pupil diameter at t₁, P₂ is the value of the pupil diameter at t₂; the rest can be done in the same manner.

Tobii T120 non-invasive eye tracker is used in preferred embodiments of the present disclosure, attention change curve when users watching pictures is recorded with 120 Hz sampling frequency.

Attention change curve generation device 3 connects with eye-movement collecting device 2. It is used for generating attention change curve based on the corresponding relationship between time sequences and pupil diameter sequences.

The attention change curve generation device 3 takes time sequences as the horizontal axis and pupil diameter sequence as a vertical axis to generate attention change curve.

The description of the working mechanism of the attention change curve generation device 3 is similar to the description mentioned above in S2 and need not be repeated here.

Numerical analysis device 4 connects with attention change curve generation device 3 which is used for numerical analysis for each stage of change curve based on time duration and pupil diameter change rate.

Five attention points are selected by numerical analysis device 4 including attention starting point, the minimum point of pupil diameter during attention process, the maximum point of pupil diameter during attention process, attention ending point and stationary point of pupil diameter after attention process

Numerical analysis device 4 set any two adjacent attention points of the five points as one stage so that the attention change curve is divided into four stages. The four attention stages are attention preparation stage, attention processing stage, attention maintaining stage and attention dissolution stage. Duration and pupil diameter change rate of four stages are calculated by numerical analysis device 4 to analyze and judge which attention stage the user is at.

The description of working mechanism of the numerical analysis device 4 is similar to the description mentioned above in S3 and need not be repeated here.

Based on the system for detecting visual attention in preferred embodiments of the present disclosure, attention process is expressed by attention change curve dynamically and systematically. The attention dynamic change and attention change curve are compared to study attention process in detail; attention regularity is portrayed more completely and systematically. While at the same time, the quantitative calculation for each stage of attention in the present disclosure portray the attention change process entirely, systematically and quantificationally which provides a new viewpoint and method for studying attention. Quota portrays, integrality and dynamic way are used to provide a new method for cognitive science and cognitive psychology research.

The preferred embodiments mentioned above are just used as examples or to explain the working mechanism of the present disclosure, but the present disclosure is not limited by it. Therefore, any amendments, equivalent replacement and improvements based on the present disclosure should be included in the protective scope. Furthermore, any transformation and amendment based on the claims should be in the protective scope of the present disclosure.

1. A method of detecting visual attention, the method comprising:
correcting time sequences and corresponding pupil diameter sequences of each regarding point during a visual attention process;
generating an attention change curve according to a corresponding relationship between the time sequences and the pupil diameter sequences;
dividing the attention change curve into four stages based on pre-setting time parameters and pupil diameter parameters; and
performing numerical analysis of each stage of the four stages based on a duration of the each stage and a rate of pupil changes during the each stage.

2. The method of claim 1, wherein the dividing the attention change curve into the four stages based on the pre-setting time parameters and the pupil diameter parameters comprises:
Selecting five attention points of the attention change curve based on the pre-setting time parameters and the pupil diameter parameters; and
dividing the attention change curve into four attention stages based on the five attention points, wherein the five attention points are:
an attention starting point,
a minimum point of a pupil diameter during the attention process,
a maximum point of the pupil diameter during the attention process,
an attention ending point, and
a stationary point of the pupil diameter after the attention process, wherein the four attention stages are:
an attention preparation stage,
an attention processing stage,
an attention maintaining stage, and
a dissolution stage.

3. The method of claim 2, wherein the numerical analysis of the attention preparation stage is performed by:
calculating an attention preparation time and an attention preparation rate using the following equations:
t_{AB}=t_{B}−t_{A},
R_{AB}=(P_{B}−P_{A})/(t_{B}−t_{A}),
wherein t_{A} represents a moment of the attention starting point, P_{A} is a value of the pupil diameter at t_{A}, t_{B} represents a moment of the attention preparation ending point, P_{B} is a value of the pupil diameter at t_{B}, t_{AB} represents the attention preparation time, and R_{AB} represents the attention preparation rate.

4. The method of claim 2, wherein the numerical analysis of the attention processing stage may be performed by:
calculating the attention processing time and the attention processing rate using the following equations:
t_{BC}=t_{C}−t_{B},
R_{BC}=(P_{C}−P_{B})/(t_{C}−t_{B}),
wherein t_{B} represents a moment of the attention preparation ending point which is a moment of the attention processing starting point, P_{B} is a value of the pupil diameter at t_{B}, t_{C} represents a moment of the attention processing ending point, P_{C} is a value of the pupil diameter at t_{C}, t_{BC} represents the attention processing time, and R_{BC} represents the attention processing rate.

5. The method of claim 2, wherein the numerical analysis of the attention maintaining stage is performed by:
calculating the attention maintaining time and the attention changing rate suing the following equations:
t_{CD}=t_{D}−t_{C},
R_{CD}=P_{CD}/(t_{D}−t_{C}),
wherein t_{C} represents a moment of the attention processing ending point which is a moment of the attention maintaining starting point, t_{D} represents a moment of the attention maintaining ending point, and t_{CD} represents the attention maintaining time,
P_{CD}=Σᵢ₌₁ⁿPᵢ/n i=1, 2, 3, . . . , n, wherein Pᵢ represents the pupil diameter of No. i attention point of the attention maintaining stage, and R_{CD} represents an attention change rate.

6. The method of claim 2, wherein the numerical analysis of the attention dissolution stage is performed by:
calculating an attention recovery time and an attention recovery rate using the following equations:
t_{DE}=t_{E}−t_{D},
R_{DE}=(P_{E}−P_{D})/(t_{E}−t_{D}),
wherein to represents a moment of the attention maintaining ending point, t_{E} represents a moment of a dissolution recovery finishing point, t_{DE} represents the attention recovery time, P_{D} is a value of the pupil diameter at t_{D}, P_{E} is a value of the pupil diameter at t_{E}, and R_{DE} represents the attention recovery rate.

7.-8. (canceled)

9. A system for detecting visual attention, the system comprising:
a stimulation providing device configured to set a visual stimulation task and provide to a user;
an eye-movement collecting device configured to collect time sequences and corresponding pupil diameter sequences of each regarding point during a visual attention;
an attention change curve generation device configured to:
connect with the eye-movement collecting device, and
generate an attention change curve based on a corresponding relationship between time sequences and pupil diameter sequences; and
a numerical analysis device configured to:
connect with the attention change curve generation device, and
perform a numerical analysis of a changing curve based on a time duration and a pupil diameter change rate, wherein the attention change curve is generated by numerical analysis device by:
selecting five attention points according to pre-setting time parameters and pupil diameter parameters;
dividing the attention change curve into four attention stages based on the five attention points;
the five attention points are:
an attention starting point,
a minimum point of the pupil diameter during an attention process,
a maximum point of the pupil diameter during the attention process,
an attention ending point, and
a stationary point of pupil the diameter after the attention process; and
the four attention stages are:
an attention preparation stage,
an attention processing stage,
an attention maintaining stage, and
an attention dissolution stage.

10. The system for claim 9, wherein the numerical analysis is performed through the numerical analysis device by:
calculating an attention preparation time and an attention preparation rate of the attention preparation stage;
calculating the attention processing time and the attention processing rate of the attention processing stage;
calculating the attention maintaining time and an attention change rate of the attention maintaining stage; and
calculating an attention recovery time and an attention recovery rate of the attention dissolution stage.
